(19) United States
(12) Patent Application Publication
(10) Pub. No.: US 2023/0256697 A1
(43) Pub. Date: Aug. 17, 2023

(54) DEVICE AND METHOD FOR SEPARATING SUBSTANCES OF DIFFERENT FLOWABILITY THAT ARE MIXED TOGETHER

(71) Applicant: NORDISCHER MASCHINENBAU RUD. BAADER GMBH + CO. KG, Lübeck (DE)

(72) Inventors: Michael Fuchs, Lübeck (DE); Andreas Tobey, Lübeck (DE); Pia Meinlschmidt, Lübeck (DE)

(21) Appl. No.: 18/015,676

(22) PCT Filed: Jun. 22, 2021
(86) PCT No.: PCT/EP2021/066893

§ 371 Date: Jan. 11, 2023

(30) Foreign Application Priority Data
Jul. 15, 2020 (DE) 10 2020 118 644.4

(51) Int. Cl. B30B 9/24; A61L 2/10

(52) U.S. Cl. CPC B30B 9/241; A61L 2/10; A22C 17/04

(57) ABSTRACT

An apparatus and method for separating substances of different flowability that are mixed together, the apparatus and method comprising a frame structure, a hollow drum driven in rotation and mounted on the frame structure and the hollow drum has a perforated circumferential surface and a partially opened side, a press belt driven in rotation and wrapped around part of the circumferential surface and can be pressed against the hollow drum, a product intake wedge configured to guide a product flow into a space between the hollow drum and the press belt in an inlet region, and a support apparatus for the press belt comprising at least one support element located on a first side of the press belt opposite the hollow drum, and a disinfection device assigned to the press belt and configured to disinfect parts of a surface of the press belt.

### CROSS-REFERENCE TO RELATED APPLICATIONS

This application is the U.S. National Stage of PCT/EP2021/066893 filed on Jun. 22, 2021, which claims priority to German Patent Application 102020118644.4 filed on Jul. 15, 2020, the entire content of both are incorporated herein by reference in their entirety.

### FIELD OF THE INVENTION

The invention relates to an apparatus configured and adapted to separate substances of different flowability that are mixed together, comprising a frame structure, a hollow drum which is driven in rotation and mounted on the frame structure and has a perforated circumferential surface and at least one at least partially open end side, an endless press belt driven in rotation in the conveying direction F which is wrapped around part of the circumferential surface of the hollow drum and can be pressed from outside against the hollow drum, a product intake wedge formed by the press belt and the hollow drum for guiding into the apparatus a product flow consisting of material to be pressed into the space between the hollow drum and the press belt in an inlet region of the product flow, a support apparatus for the press belt with at least one support element which is located on the side of the press belt opposite the hollow drum.

The invention further relates to a method for separating substances of different flowability that are mixed together, comprising the following steps: Feeding a product flow, consisting of material to be pressed, into a product intake wedge which is formed by a hollow drum with a perforated circumferential surface and a press belt which lies against the outside of the hollow drum while wrapping around part of the circumference, driving the hollow drum and/or the press belt in rotation in order to draw the product flow between the hollow drum and the press belt, the easier-flowing constituents of the product flow being pressed by means of the press belt as separation material through the perforations of the circumferential surface of the hollow drum into the hollow space of the hollow drum and being discharged from an at least partially opened end side of the hollow drum, while the harder-flowing constituents of the product flow remain outside the circumferential surface of the hollow drum and are discharged separately.

### BACKGROUND OF THE INVENTION

Such apparatuses and methods are used in particular in the food processing industry to separate substances, materials or products that differ, or whose composition differs, in terms of their flow behaviour. The possibility and/or necessity of separating substances of different flowability mixed together exists in particular in the production of foodstuffs of animal origin (for example, separation of meat from skin, sinews, bones, fishbones etc.), foodstuffs made from fruit and vegetables (for example, separation of fruit flesh from skin, stalks, seeds, stones etc.), and in the processing of incorrectly packaged foodstuffs (for example, separation of butter or cheese from packaging material) and the like. Specific application examples include, for example, coring/pitting of dates or unpacking of cheese slices wrapped in film.

During utilisation, coring, unpacking or any other corresponding separation process, a product flow is conveyed from a product feed area, the inlet region, with a product intake wedge between the press element, for example the press belt, and the hollow drum, to a product discharge area, the outlet region. The product intake wedge is formed by the arrangement of the press belt relative to the hollow drum and creates an (intake) angle in the inlet region where the product flows into the apparatus. Due to the rotating drive of the hollow drum and/or the rotating drive of the press belt, the product flow is drawn in between the hollow drum and the press belt in the inlet region in the conveying direction F, so that a distance/gap (filled with product flow) is created between the press belt and the hollow drum. Downstream of the product intake wedge (in relation to the conveying direction of the product flow), a pressing region is formed between the press belt and the hollow drum by the wrap-around, through which the product flow is conveyed. The substances of different flowability are separated by pressing the easier-flowing substances, in the example of processing products of animal origin the meat, at least partially as separation material, which in the example of products of animal origin consists of small meat threads or small meat strands, into the hollow drum and discharging them from there. Discharge takes place via an at least partially open end side of the hollow drum, for example on the front side of the apparatus, it being also possible to define the front side as the rear side. The harder-flowing materials, which again using the example of products of animal origin consist of bones, sinews, skin, etc., are released from the press belt downstream of the hollow drum in relation to the conveying direction F of the product flow in the product discharge area, then collected and discharged.

The press belt, which is substantially freed from residual product flow, then continues to revolve in order to be refilled/resupplied with product flow in the inlet region. In other words, an upper run of the press belt carries product, while a lower run is returned without product. However, without product does not mean that the surface of the revolving press belt facing the hollow drum in the area of the upper run is clean and germ-free. Due to the intake of animal and/or vegetable products in particular, germs can also enter the apparatus with these products. Pressing germ-laden products between the press belt and the hollow drum can then, among other things, cause the press belt to become germ-laden, which can lead to cross-contamination when the press belt continues to revolve and subsequent products are fed into the product intake wedge. It is also possible for germs to form in the apparatus due to chemical reactions between product residues and the ambient oxygen. Germ formation has a detrimental effect on product quality overall. There is a risk that regular cleaning and/or disinfection, for example after each production shift, will not adequately remedy the described problems that arise in particular due to the processing of germ-laden products.

### SUMMARY OF THE INVENTION

The invention is thus based on the task of proposing a simple and reliable apparatus that enables an efficient separation process as well as improved product quality. The task further consists in proposing a corresponding method.

This task is solved by an apparatus of the type mentioned hereinbefore wherein a device is assigned to the press belt that is configured and adapted to disinfect at least parts of the surface of the press belt during operation of the apparatus. The disinfection device can optionally be used to disinfect a part of the surface of the press belt or the entire surface of the press belt. Disinfection is provided in particular on the surface facing towards the hollow drum. Disinfection can also be carried out by means of the disinfection device on the surface of the press belt facing away from the hollow drum, that is to say, on the back of the belt. The inventive design of the apparatus ensures a reduced germ load on the surface of the press belt and thus of the product during operation of the apparatus. In particular, the risk of cross-contamination can be reduced. The fact that disinfection can be carried out continuously means that the apparatus can be operated with shorter downtimes. Continuous reduction of the germ load of the press belt and thus of the product with human-pathogenic and product-damaging germs, such as Salmonella, Listeria monocytogenes, Escherichia coli, lactic acid bacteria and the like, improves product quality in terms of the germ load.

Advantageously, the frame structure is configured as a housing which comprises a closed working space accommodating at least the hollow drum, the press belt and the support apparatus, which working space has, at least on one side, a flap which can be opened and closed and which covers the working space with the exception of the at least partially open end side S_{V} of the hollow drum. Since the device for disinfecting the press belt is dedicated to the press belt, the disinfecting device is also located inside the housing, so that a person operating the apparatus is effectively protected against adverse effects of UV radiation emanating from the disinfecting device. For this purpose, the housing is at least partially made of a UV light-absorbing material or is shielded by appropriate covers, shields or similar in such a way that no harmful UV rays can emerge from the housing.

A preferred embodiment is characterised in that the press belt has an upper run and a lower run, the upper run forming a pressing region as a pressing run with the hollow drum, and the lower run forming a return run from the outlet region A to the inlet region E, the disinfection device being disposed in the area of the lower run. Surface disinfection of the press belt can be carried out particularly easily and directly in the lower run. The disinfection device is preferably located underneath the lower run. However, it is also possible that the disinfection device or at least parts thereof are located between the upper run and the lower run in order to also disinfect the back of the press belt, which does not face the hollow drum, in the area of the lower run.

The device expediently comprises a device for discharging separation material pressed through the perforated circumferential surface into the hollow space of the hollow drum from the at least partially open end side of the hollow drum. The hollow drum itself can be the device for discharging, for example by giving a suitable shape to the inner surface of the hollow drum. Optionally, however, a separate device may also be provided, for example in the form of an ejection screw or a stripping element.

A particularly advantageous further development is characterised in that the disinfection device comprises at least one UV lamp, the or each UV lamp being disposed inside the housing and shielded from the environment. For the purposes of the invention, a UV lamp is a device for emitting electromagnetic radiation in the wavelength range of ultraviolet light. With the or each UV lamp, it is possible to disinfect the surface of the press belt from human pathogenic and resistant germs by approximately 90%. The device may comprise one, two or more than two UV lamps. The number of UV lamps and their positioning along the press belt and/or their orientation can vary. This continuous disinfection accompanying the separation process may possibly reduce the downtimes of the apparatus and significantly extend the intervals between cleaning and disinfection with the apparatus at a standstill. The fact that each UV lamp is disposed in the housing ensures that no UV rays escape to the outside.

Particularly preferably, the disinfection device comprises at least one UV lamp pair. Particularly intensive and effective disinfection can be realised with the or each lamp pair. Because the entire width of the press belt can be exposed to UV light applied transverse to the conveying direction of the press belt with one lamp pair and a large amount of light can be applied to the press belt, it is possible to disinfect the surface of the press belt from human pathogens and resistant germs by around 99%. This continuous disinfection accompanying the separation process can further reduce the downtimes of the apparatus and still further extend the intervals between cleaning and disinfection with the apparatus at a standstill.

Advantageously, the or each UV lamp is configured as a UV(C) lamp with an elongated lamp body which is configured and adapted to generate light in the wavelength range from 280 nm to 100 nm. Particularly reliable and effective disinfection is guaranteed with these so-called UV(C) lamps. The elongated, tubular lamp body can be aligned transverse to the conveying direction of the press belt or parallel to it. The shape and design of the lamp bodies is not linked to the design of the UV lamp as a UV(C) lamp. Other shapes and designs of lamp bodies and other arrangements and orientations thereof in relation to the conveying direction can also be used.

Particularly preferably, the distance of the or each UV lamp—and thus also of each UV(C) lamp—from the press belt is at least approximately 0.5 to 10 cm, further preferably 1 to 8 cm and particularly preferably 2 to 6 cm. Particularly effective disinfection is guaranteed with a UV lamp pair in which the two UV(C) lamps are aligned parallel to and spaced apart from one another and in which the two UV(C) lamps are at a distance of 2 to 4 cm from the conveyor plane spanned by the press belt.

As mentioned, the UV lamps or UV(C) lamps of a UV lamp pair can optionally be aligned parallel to or converging with one another. The or each UV lamp or UV(C) lamp can be oriented in its longitudinal extension substantially transverse to the conveying direction. For this purpose, two or more UV lamps or UV(C) lamps spaced apart from one another can form one UV lamp unit. Such units, which can be configured, for example, as an interchangeable module, can then be used at different positions along the press belt and in particular the lower run. A plurality of such units may also be used. In other configurations, the or each UV lamp or UV(C) lamp is oriented in its longitudinal extension substantially parallel to the conveying direction of the press belt.

In a particularly preferred embodiment, at least two UV lamp pairs are provided, with a first UV lamp pair being disposed underneath the press belt on the side opposite the hollow drum and the second UV lamp pair being disposed laterally underneath the press belt below the product intake wedge. UV lamp pairs also include UV(C) lamp pairs. In other words, a first disinfection takes place in the area of the lower run on the side opposite the pressing region and a further disinfection takes place in the area of the lower run before it again becomes the upper run in the inlet region. This creates an apparatus which is compact and very efficient as regards disinfection of the press belt.

Optionally, at least three UV lamp pairs are provided, distributed along the lower run starting from an outlet region as far as the inlet region.

An advantageous further development is characterised in that the apparatus comprises a control and/or regulation device to which at least the disinfection device and a contact switch of the flap are connected, such that the or each UV lamp can be automatically switched off when the flap of the housing is opened. As soon as the light is no longer sealed off by the housing or the flap, the disinfection device switches off automatically. This ensures safety in the workplace with regard to UV radiation. Instead of a contact switch, other safety systems can also be used to ensure that the power supply to the or each UV lamp is interrupted as soon as light from the housing is no longer adequately sealed off. In other words, each UV lamp is switched off as soon as there is a risk that UV rays could radiate into the environment of the apparatus. Other components of the apparatus, for example the drive means for the hollow drum and/or the press belt, can also be connected to the control and/or regulating device.

The task is also solved by a method with the steps mentioned hereinbefore in that the press belt is disinfected during the separation process at least in the area of parts of the surface. The separating and disinfecting processes are thus carried out in parallel. On the one hand, this leads to significantly improved efficiency and performance of the apparatus due to shorter downtimes and longer intervals between cleaning and disinfection. On the other hand, online disinfection leads to improved product quality, as disinfection starts earlier and is carried out regularly and continuously.

Preferably, the product flow is fed into an inlet region of an apparatus for separating substances of different flowability that are mixed together, while the separation material that has been pressed into the hollow drum is discharged from the at least partially open end side and the harder-flowing constituents of the product flow are detached from the press belt in the area of an outlet region and discharged, at least parts of the surface of the press belt being exposed to ultraviolet light (UV light) as it is led back from the outlet region to the inlet region.

Particularly preferably, the press belt or its surface is exposed to ultraviolet light (UV light) in the wavelength range from 280 nm to 100 nm.

Expediently, the UV light is continuously directed onto the press belt during the separation process. This means that the press belt is inevitably exposed to UV light as it is led back, so that the press belt is always disinfected over its entire length during operation.

In a preferred further development, the separation process is carried out in a substantially closed working space of the apparatus, the UV light being automatically switched off as soon as the working space is opened. The UV light can thus only be emitted in a protected, sealed-off environment, namely the enclosed working space.

More particularly preferably, the method is carried out with an apparatus as disclosed herein.

Other advantages resulting from the aforementioned process steps have already been described in relation to the apparatus, therefore reference is made to the above explanations so as to avoid repetition.

### BRIEF DESCRIPTION OF THE DRAWINGS

Further useful and/or advantageous features and further developments of the apparatus and the method can be seen in the description. Particularly preferred embodiments of the apparatus and method are explained in more detail with reference to the attached drawing. The drawing shows the following:

FIG. 1A schematic representation of the apparatus according to the invention in a perspective view with the front flap closed;

FIG. 2A schematic representation of the apparatus according to FIG. 1 without the front flap in a perspective view; and

FIG. 3A schematic representation of a section of the illustration according to FIG. 2 in a further perspective, viewed obliquely from below.

### DETAILED DESCRIPTION OF THE INVENTION

The apparatus shown in the drawing is used for separating meat on the one hand from bones, bone residues, sinews, gristle and the like on the other hand and has a plurality of preferred features. Other embodiments having fewer or different features are not explicitly shown. The device can also be used in the same way for separating, for example, fruit flesh from seeds or for separating any other mixed substances of different flowability.

The apparatus 10 shown is configured and adapted to separate substances of different flowability that are mixed together. The apparatus 10 comprises a frame structure 11, a hollow drum 12 which is driven in rotation and mounted on the frame structure 11 and has a perforated circumferential surface M and at least one at least partially open end side S_{V}, S_{H}, an endless press belt 13 driven in rotation in the conveying direction F which is wrapped around part of the circumferential surface M of the hollow drum 12 and can be pressed from outside against the hollow drum 12, a product intake wedge 14 formed by the press belt 13 and the hollow drum 12 for guiding into the apparatus 10 a product flow consisting of material to be pressed into the space between the hollow drum 12 and the press belt in an inlet region E of the product flow, and a support apparatus 15 for the press belt 13 with at least one support element 16 which is located on the side of the press belt 13 opposite the hollow drum 12.

The end side S_{V} of the hollow drum 12 facing the front side is open, while the opposite end side S_{H} is preferably closed. The front and back sides may also be reversed.

This apparatus 10 is characterised, according to the invention, in that a device 17 is assigned to the press belt 13 that is configured and adapted to disinfect at least parts of the surface of the press belt 13 during operation of the apparatus 10. The device 17 is therefore an integral part of the apparatus 10 and enables disinfection during operation, that is to say, during the separation process.

The features and further developments described below represent preferred embodiments, either individually or in combination with each other. It is expressly pointed out that features which are summarised in the description and/or the drawing or described in a common embodiment can also functionally and independently further form the apparatus 10 described above.

The frame structure 11 can be a simple profile construction. The frame structure 11 is preferably a housing 18 that is substantially closed on all sides. The drive unit (not shown) for the hollow drum 12 and/or the press belt 13 is disposed on or in the frame structure 11. The drive unit may comprise a common drive or separate drive means. The rotation axis R of the hollow drum 12 runs transverse to the conveying direction F of the press belt 13 and also of the product flow. Holes/openings (not explicitly shown) are formed in the circumferential surface M of the hollow drum 12, which can be disposed in different patterns and allow the passage of parts of the product flow, in particular the easier-flowing, soft and squeezable parts, into the interior of the hollow drum 12. The hollow drum 12 is preferably provided with such openings over its entire depth or width, which forms the working area. The press belt 13 is preferably made of an elastic material, for example rubber, polyurethane or similar, and is guided around a plurality of deflection elements 19. Preferably, a deflection element 19 is configured and adapted as a drive roller 20. Particularly preferably, the deflection element 19, which is placed downstream of the hollow drum 12 in the conveying direction F, is the drive roller 20, in order to guide the press belt 13 and thus the product flow past the hollow drum 12 with a pulling motion. Particularly preferably, the drive roller 20 is simultaneously configured and adapted as a pressure roller and/or tension roller for the press belt 13. For this purpose, the drive roller 20 is configured so as to be adjustable. The conveying of the product flow in the conveying direction F can be further supported by the hollow drum's 12 own drive.

Optionally, stripping elements can be disposed in the inlet region E, that is to say, above the product intake wedge 14, and/or in the outlet region A, that is to say, downstream of the hollow drum 12, also referred to as the perforated drum, in the conveying direction F. These stripping elements can be disposed rigidly, that is to say, at a fixed distance from the hollow drum 12 or, more precisely, from the circumferential surface M, or they can be variably controllable, that is to say, their distance from the hollow drum 12 or, more precisely, from the circumferential surface M can be varied. The hollow drum 12 and the press belt 13 can be driven at the same speed. Optionally, however, the hollow drum 12 and the press belt 13 can also be driven at different speeds. The speed difference can be controlled and/or regulated by means of a control and/or regulating device 21. The support apparatus 15 can be a support chain or a support belt. In the embodiment shown, the support apparatus 15 is configured as a roller track 22 comprising a plurality of rollers disposed one after the other in the conveying direction F of the product flow and at least partially spring-mounted.

Particularly preferably, the frame structure 11 is configured as a housing 18 which comprises a closed working space 23 accommodating at least the hollow drum 12, the press belt 13 and the support apparatus 15 and has, at least on one side, a flap 24 which can be opened and closed and covers the working space 23 with the exception of the at least partially open end side S_{V} of the hollow drum 12. The disinfection device 17 is also located within the working space 23. The flap 24 may constitute a front flap of the housing 18. In the closed state, the front flap enables the working space 23 to be sealed off from the environment. In the open state, out of operation, accessibility is guaranteed for cleaning and/or maintenance purposes. However, the housing 18 may also have other closable openings and access points in other positions.

The circulating press belt 13 has an upper run 25 and a lower run 26, the upper run 25 forming a pressing region P as a pressing run with the hollow drum 12, and the lower run 26 forming a return run from the outlet region A to the inlet region E, the disinfection device 17 being disposed in the area of the lower run. Due to the rotating drive of the press belt 13, the upper run 25 is alternately also the lower run 26 and vice versa. In the upper run 25, the surface of the press belt 13 to be disinfected faces towards the hollow drum 12, while in the lower run 26, the surface of the press belt 13 to be disinfected faces downwards/outwards.

The separation material pressed through the circumferential surface M can be discharged from the hollow drum 12 of its own accord, solely through the rotation of the drum or the shaping of its inner surface. In this case the hollow drum 12 itself forms a device for discharging the separation material. The device for discharging the separation material from the hollow drum 12 from the at least one at least partially open end side S_{V}, S_{H} preferably comprises a stripping element and/or an ejection screw. The discharged separation material can, for example, fall onto a discharge channel or similar which can optionally be cooled and is connected to a corresponding device for this purpose. The press belt 13 then runs substantially empty from the outlet region A back to the inlet region E. In this region, the disinfection device 17 has a disinfecting effect by acting directly on at least sections of the surface of the press belt 13.

Optionally, the disinfection device 17 comprises at least one UV lamp 27, the or each UV lamp 27 being disposed inside the housing 18 and shielded from the environment. When the apparatus 10 is in operation, the housing 18 is sealed off so that no UV rays escape to the outside. For this purpose, the housing 18 may be made entirely or partially of UV-absorbing material. Covers, screens or similar can optionally also be provided to prevent UV rays from escaping. The position of the or each UV lamp 27 may vary. A single UV lamp 27 may be disposed in the area of the lower run 26 immediately in front of the inlet region E. In other embodiments, two UV lamps 27 are provided. A UV lamp 27 can be disposed in the area of the lower run 26 immediately before the inlet region E. In addition, a second UV lamp 27 may be disposed in the area of the lower run 26 underneath the hollow drum 12. The or each UV lamp 27 can also be replaced by a UV lamp pair. Particularly preferably, at least two UV lamp pairs are provided, with a first UV lamp pair being disposed underneath the press belt 13 on the side opposite the hollow drum 12 and the second UV lamp pair being disposed laterally underneath the press belt 13 below the product intake wedge 14.

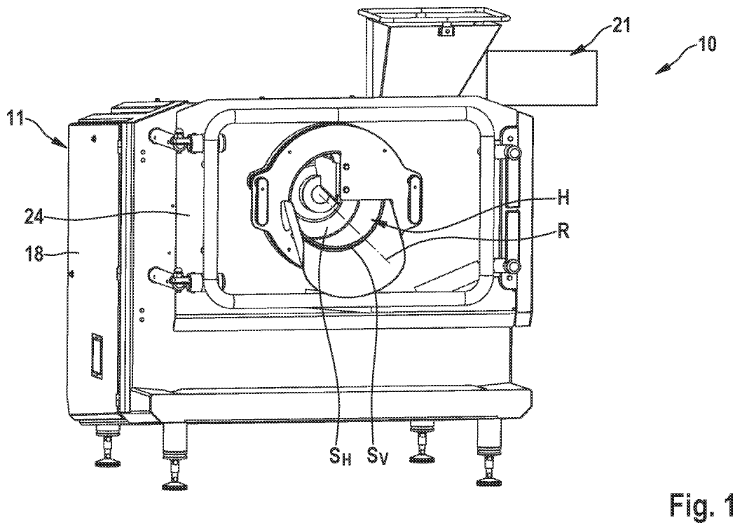

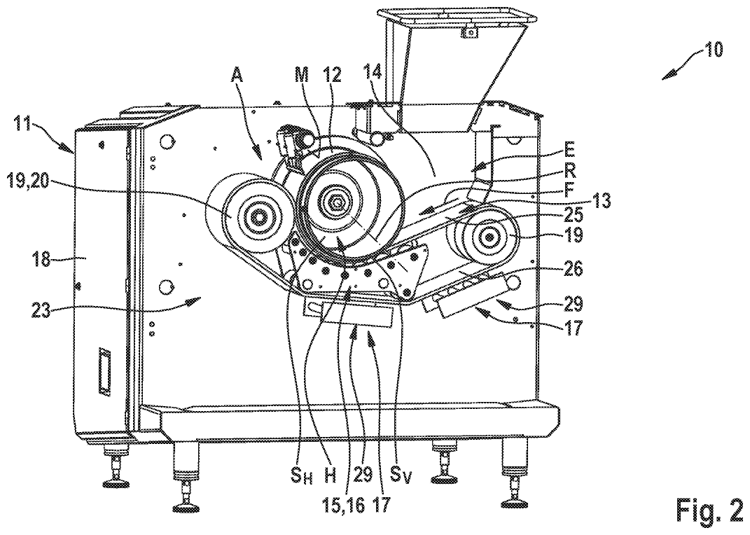

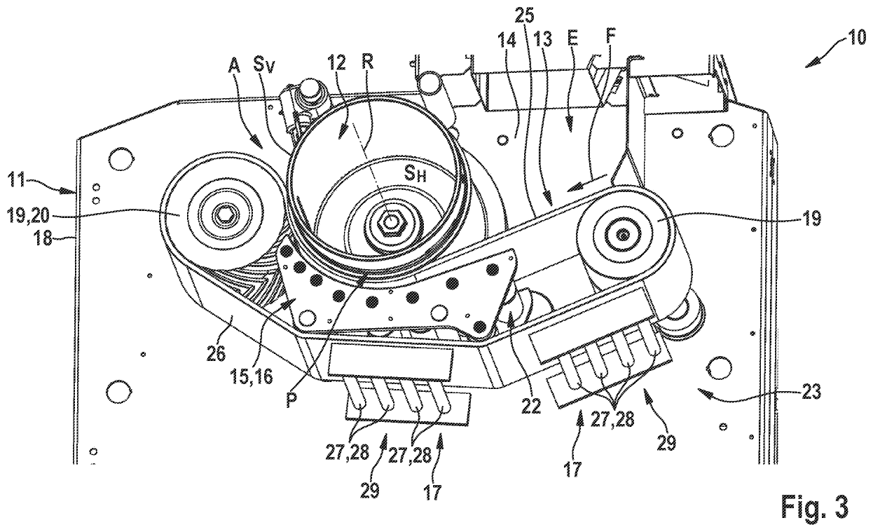

Preferably and as shown in the drawing, the or each UV lamp 27 is configured as a UV(C) lamp with an elongated lamp body 28 which is configured and adapted to generate light in the wavelength range from 280 nm to 100 nm. The shape of the lamp bodies 28 can also vary when using UV(C) lamps. The distance of the or each UV lamp 27 from the press belt is at least approximately 0.5 to 10 cm, further preferably 1 to 8 cm and particularly preferably 2 to 6 cm. However, other distances can also be chosen. The UV lamps 27 of a UV lamp pair are optionally aligned parallel to or converging with each other. In embodiments not explicitly shown, the or each UV lamp 27 is oriented in its longitudinal extension substantially parallel to the conveying direction F of the press belt 13. In the embodiment according to FIGS. 1 to 3, the or each UV lamp 27 is oriented in its longitudinal extension substantially transverse to the conveying direction F. In this case, four lamp bodies 28 each form a lamp unit 29, two of which are positioned in the area of the lower run 26. Optionally, for example at least three UV lamp pairs are provided, distributed along the lower run 26 starting from an outlet region A as far as the inlet region E. Furthermore, a UV lamp 27, a UV lamp pair or at least one lamp unit 29 can also be positioned between the upper run 25 and the lower run 26.

As already described, the apparatus 10 comprises a control and/or regulating device 21. At least the disinfection device 17 and a contact switch (not explicitly shown) of the flap 24 are connected to this control and/or regulating device 21, such that the or each UV lamp 27 is or can be automatically switched off when the flap 24 of the housing 18 is opened. By means of the control and/or regulating device 21, it can be ensured that at least any opening of the housing 18 that could lead to UV rays escaping results in each UV lamp 27 being switched off.

The method is described below in more detail with reference to the drawing. The method serves, and is accordingly configured and adapted, to separate substances of different flowability that are mixed together. First, a product flow, consisting of material to be pressed, is fed into a product intake wedge 14 formed by a hollow drum 12 with a perforated circumferential surface M and a press belt 13 which lies against the outside of the hollow drum 12 while wrapping around part of its circumference. For the separation process, the hollow drum 12 and the press belt 13 are driven in rotation to draw the product flow between the hollow drum 12 and the press belt 13. Preferably, the hollow drum 12 (in the view according to FIGS. 1 to 3) is driven clockwise, while the drive roller 20 for the press belt (in the view according to FIGS. 1 to 3) is driven counterclockwise, so that the hollow drum 12 and the press belt 13 have the same conveying direction F in the area of the wrap-around, that is to say, in the pressing region P, the transport speeds being the same as or different to each other. By transporting the product flow through the pressing region P, the easier-flowing constituents of the product flow are pressed by means of the press belt 13 as separation material through the perforation of the circumferential surface M of the hollow drum 12 into the inner hollow space H of the hollow drum 12 and discharged from an at least partially opened end side S_{V} and/or S_{H} of the hollow drum 12, while the harder-flowing constituents of the product flow remain on the outside of the circumferential surface M of the hollow drum 12 and are discharged separately. The harder-flowing constituents are discharged in the outlet region A.

This method according to the invention is characterised in that the press belt 13 is disinfected during the separation process at least in the area of parts of the surface. Preferably, the product flow is fed into an inlet region E of an apparatus 10 for separating substances of different flowability that are mixed together, while the separation material which has been pressed into the hollow drum 13 is discharged from the at least partially open end side S_{V}, S_{H} and the harder-flowing constituents of the product flow are detached from the press belt 13 in the area of an outlet region A and discharged, at least parts of the surface of the press belt 13 being exposed to ultraviolet light (UV light) as it is led back from the outlet region A to the inlet region E, so that the product flow which is newly fed to the product intake wedge 14 meets a substantially disinfected press belt 13.

The press belt 13 or its surface is preferably exposed to ultraviolet light (UV light) in the wavelength range from 280 nm to 100 nm. The UV light can be temporarily directed onto the press belt 13. Preferably, the UV light is continuously directed onto the press belt 13 during the separation process, so that the surface is lastingly and permanently exposed to the disinfecting effect of the UV light.

Expediently, the separation process is carried out in a substantially closed working space 23 of the apparatus 10, the UV light being automatically switched off as soon as the working space is 23 opened. The disinfection device 17 and thus in particular each UV lamp is sealed off from the environment so that radiation from the UV lamps 27 cannot penetrate to the outside. The power supply to the UV lamps 27 is interrupted as soon as the protected working space 23 is opened.

During separation of the product flow into its constituents, the press belt 13 is preferably supported on the side of the press belt 13 opposite to the hollow drum 12 by a support apparatus 15 comprising at least one support element 16.

More particularly preferably, the method is carried out with an apparatus 10 according to one or more of claims 1 to 12.

1-20. (canceled)

21. An apparatus for separating substances of different flowability that are mixed together, the apparatus comprising:
a frame structure,
a hollow drum, wherein:
the hollow drum is driven in rotation and mounted on the frame structure; and
the hollow drum has a perforated circumferential surface and at least one end side, wherein the at least one end side is at least partially open,
an endless press belt driven in rotation in a conveying direction F, wherein:
the endless press belt is wrapped around part of the circumferential surface of the hollow drum; and
the endless press belt can be pressed from outside against the hollow drum,
a product intake wedge formed by the endless press belt and the hollow drum, wherein the product intake wedge is configured to guide, into the apparatus, a product flow consisting of material to be pressed, wherein the product flow is guided into a space between the hollow drum and the endless press belt in an inlet region, and
a support apparatus for the endless press belt, the support apparatus comprising at least one support element, wherein:
the support element is located on a first side of the endless press belt;
the first side is opposite the hollow drum; and
a disinfection device is assigned to the endless press belt, wherein the disinfection device is configured to disinfect at least parts of a surface of the endless press belt during operation of the apparatus.

22. The apparatus of claim 21, wherein the frame structure comprises a housing, wherein:
the housing comprises a closed working space,
the closed working space accommodates at least the hollow drum, the endless press belt, and the support apparatus,
the closed working space comprises, on at least one side of the closed working space, a flap,
the flap can be opened and closed, and
the flap covers the working space with the exception of the at end side of the hollow drum.

23. The apparatus of claim 21, wherein:
the endless press belt comprises an upper run and a lower run,
the upper run forms a pressing region as a pressing run with the hollow drum,
the lower run forms a return run from an outlet region to the inlet region, and
the disinfection device is disposed in an area of the lower run.

24. The apparatus of claim 21, wherein the apparatus further comprises a device for discharging separation material pressed through the perforated circumferential surface into a hollow space of the hollow drum from the end side of the hollow drum.

25. The apparatus of claim 21, wherein:
the disinfection device comprises at least one UV lamp,
the at least one UV lamp is disposed inside the housing, and
the at least one UV lamp is shielded from the environment.

26. The apparatus of claim 22, wherein the disinfection device comprises at least one UV lamp pair.

27. The apparatus of claim 25, wherein:
the at least one UV lamp comprises a UVC lamp,
the UVC lamp comprises an elongated lamp body, and
the elongated lamp body is configured to generate light in the wavelength range from 280 nm to 100 nm.

28. The apparatus of claim 25, wherein a distance from the at least one UV lamp to the endless press belt is at least approximately 0.5 to 10 cm, more

[symbol]

29. The apparatus of claim 28, wherein the distance is 1 to 8 cm.

30. The apparatus of claim 29, wherein the distance is 2 to 6 cm.

31. The apparatus of claim 26, wherein a first UV lamp of the UV lamp pair is aligned either:
parallel to a second UV lamp of the UV lamp pair, or
converging with the second UV lamp of the UV lamp pair.

32. The apparatus of claims 26, wherein:
the apparatus comprises at least two UV lamp pairs,
a first UV lamp pair is disposed underneath the endless press belt on the first side, and
a second UV lamp pair is disposed laterally underneath the endless press belt and below the product intake wedge.

33. The apparatus of claim 26, wherein:
the apparatus comprises at least three UV lamp pairs,
the lamp pairs are distributed along a lower run of the endless press belt, from an outlet region to the inlet region.

34. The apparatus of claim 21, wherein
the apparatus further comprises a control and/or regulation device,
at least the disinfection device and a contact switch of the flap are connected to the control and/or regulation device, and
the at least one UV lamp can be automatically switched off when the flap of the housing is opened.

35. A method for separating substances of different flowability that are mixed together, the method comprising:
feeding a product flow consisting of material to be pressed, wherein the product flow is fed into a product intake wedge, wherein the product intake wedge is formed by a hollow drum with a circumferential surface, the circumferential surface comprising a perforation, and a press belt, wherein the press belt lies against an outside of the hollow drum while wrapping around part of a circumference of the hollow drum,
driving at least one of the hollow drum and the press belt in a rotation, wherein the rotation is configured to draw the product flow between the hollow drum and the press belt,
easier-flowing constituents of the product flow are pressed by the press belt as separation material in a separation process:
through the perforation of the circumferential surface of the hollow drum;
into a hollow space of the hollow drum; and
discharged from an end side of the hollow drum, wherein the end side comprises at least one opening,
harder-flowing constituents of the product flow remains on the outside of the hollow drum and are discharged separately, and
the press belt is disinfected during the separation process at least in parts of a surface of the press belt.

36. The method of claim 35, wherein:
the product flow is fed into an inlet region of an apparatus for separating substances of different flowability that are mixed together,
the separation material, after being pressed, is discharged from the opening of the end side,
the harder-flowing constituents of the product flow are detached from the press belt in an outlet region and discharged, and
at least an exposed part of the surface of the press belt is exposed to ultraviolet light as the exposed part is led back from the outlet region to the inlet region.

37. The method of claim 35, wherein the press belt or a surface of the press belt is exposed to ultraviolet light in the wavelength range from 280 nm to 100 nm.

38. The method of claim 36, wherein the ultraviolet light is continuously directed onto the press belt during the separation process.

39. The method of claim 36, wherein:
the separation process is carried out in a substantially closed working space of the apparatus, and
the ultraviolet light is automatically switched off as soon as the working space is opened.

40. The method of claim 35, the method is carried out with an apparatus for separating substances of different flowability that are mixed together, the apparatus comprising:
a frame structure,
a hollow drum, wherein:
the hollow drum is driven in rotation and mounted on the frame structure; and
the hollow drum has a perforated circumferential surface and at least one end side, wherein the at least one end side is at least partially open,
an endless press belt driven in rotation in a conveying direction F, wherein:
the endless press belt is wrapped around part of the circumferential surface of the hollow drum; and
the endless press belt can be pressed from outside against the hollow drum,
a product intake wedge formed by the press belt and the hollow drum, wherein the product intake wedge is configured to guide, into the apparatus, a product flow consisting of material to be pressed, wherein the product flow is guided into a space between the hollow drum and the press belt in an inlet region of the product flow, and
a support apparatus for the press belt, the support apparatus comprising at least one support element, wherein:
the support element is located on a first side of the press belt;
the first side is opposite the hollow drum; and
a disinfection device is assigned to the press belt, wherein the disinfection device is configured to disinfect at least parts of a surface of the press belt during operation of the apparatus.
